(19) United States
(12) Patent Application Publication
(10) Pub. No.: US 2012/0271353 A1
(43) Pub. Date: Oct. 25, 2012

(54) SYSTEM AND METHOD FOR ALIGNING VERTEBRAE IN THE AMELIORATION OF ABERRANT SPINAL COLUMN DEVIATION CONDITIONS IN PATIENTS REQUIRING THE ACCOMODATION OF SPINAL COLUMN GROWTH OR ELONGATION

(76) Inventor: Mark Barry, Las Vegas, NV (US)

(21) Appl. No.: 12/980,737

(22) Filed: Dec. 29, 2010

Related U.S. Application Data
(63) Continuation-in-part of application No. 12/857,320, filed on Aug. 16, 2010.

(51) Int. Cl. A61B 17/70

(52) U.S. Cl. 606/258; 606/265

(57) ABSTRACT

A system and method for ameliorating spinal column anomalies, such as scoliosis, while accommodating growth of juvenile patients, includes pedicle screws and an extendable telescopic spinal rod of non-circular cross section.

### CITATION TO PARENT APPLICATION

This is a continuation-in-part application which respect to co-pending U.S. application Ser. No. 12/857,320, filed 16 Aug. 2010, from which priority is claimed.

### BACKGROUND OF THE INVENTION

1. Field of the Invention

The present invention relates to methods and apparatus for management and correction of spinal deformities, such as scoliosis.

2. Background Information

A serious deficiency presently exists with respect to conventional treatment and instrumentation for treating spinal deviation anomalies (such as scoliosis). This is particularly true as it relates to juvenile (age 3-10) cases involving greater than 45° curvatures (as such terminology is understood in the field) and more particularly to idiopathic scoliosis.

Currently, idiopathic scoliosis (“I.S.”) comprises approximately 75% of all juvenile cases. Those I.S. cases involving curvatures in the 25°-45° range indicate treatment through bracing (beginning roughly at the bottom end of this range), but become unbeatable by bracing (roughly at the top end of this range). Curvatures in excess of 45° indicate surgical intervention.

Use of implanted spinal rod systems of the current art introduce significant patient risks. These include considerable likelihood of hardware dislodgement (such as when hooks are used to engage spinal rod system components), ulcerations of skin that overlies protrusions of implanted systems, premature fusion of adjacent vertebrae with highly deleterious growth and spinal contour issues, impairment of longitudinal spinal growth, worsening of axial plane deformities such as rib hump, aggravation of truncal balance problems, and greater chance of infections.

To make matters worse, existing spinal rod systems, particularly when used in juveniles, require periodic lengthening and adjusting to accommodate growth (roughly every 6-9 months). For growing patients, especially juveniles, periodic lengthening and adjusting accommodates the change or increase in distance between spinal segments. Multiple surgical procedures may be required to adjust one or more components for lengthening and adjusting the spinal device. Further still, the existing systems only control curvature in two dimensions. Finally, a formal fusion procedure is required at or near skeletal maturity.

An ideal system for addressing the present shortcomings of treatment options for juvenile scoliosis involving greater than 45° curvatures is one which (at least): (1) provides three-dimensional correction of spinal anomalies; (2) provides secure engagement between instrumentation of affected vertebrae; (3) obviates or diminishes the need for periodic lengthening procedures; and (4) obviates the need for formal fusions at skeletal maturity.

Such a system would only be possible were it to “grow” with the patient (accommodate changes in distance in spinal segments or vertebrae), utilize other than easily dislodgeable skeletal engagement means, and maintain desired orientation and alignment of vertebrae in all dimensions.

With respect to this latter objective: current spinal rods are of circular or round cross section. Were present spinal rods or attachment means to be left “loose” to accommodate longitudinal motions as vertebrae move relatively as a result of growth, there would be nothing to combat the deleterious axial rotation of the vertebrae (relative to the spinal rod) even as they are constrained in their longitudinal movement along the rod. Such axial rotation would result in far less than optimal correction of the overall spinal geometry, and potentially impair longitudinal growth of the spine.

Were an ideal system for addressing juvenile scoliosis requiring surgical intervention to become available (addressing each of the above-listed shortcomings of the systems and methods of the present art), the recipients would benefit in at least the following ways: (1) they would enjoy a much higher incidence and degree of success in alleviating their spinal deformities (in all dimensions of spinal column geometry); (2) they would achieve more nearly normal growth expectations; (3) they would be spared from multiple surgical procedures with their associated risks and complications; (4) they would not face the painful and potentially catastrophic consequences of spinal rod system component dislodgement; and (5) they would maintain mobility at adulthood that would otherwise be lost though otherwise required fusions.

### SUMMARY OF THE INVENTION

In view of the foregoing, it is an object of certain embodiments of the present system to provide an improved system of spinal instrumentation for use in ameliorating aberrant spinal column deviation conditions, such as scoliosis, particularly (though not necessarily solely) in juvenile cases of idiopathic scoliosis.

It is another object of certain embodiments of the present system to provide an improved system and associated method for ameliorating aberrant spinal column deviation conditions, such as scoliosis, which system and method addresses each of the above-listed shortcomings of the spinal rod systems and methods for addressing juvenile scoliosis that is of the present art.

It is another object of certain embodiments of the present system to provide an improved system and associated method for ameliorating aberrant spinal column deviation conditions, such as scoliosis, which system and method reduce hazards to patients relating at least to implantation of instrumentation, subsequent post-implantation surgical interventions related to accommodation of patient growth, spontaneous vertebral fusions, and inhibition of normal growth of the spine.

It is another object of certain embodiments of the present system to provide an improved method for ameliorating aberrant spinal column deviation conditions, such as scoliosis, which system accommodates growth without surgical intervention to the degree required of spinal rod systems of the present art.

It is another object of the present system to provide an improved system of spinal instrumentation, and a method for the use thereof, for ameliorating aberrant spinal column deviation conditions, such as scoliosis, which system and method facilitate maintaining spinal correction in three dimensions, rather than the merely two dimensions presently achievable (to a limited degree, and with limited success) with systems and methods of the present technology.

In satisfaction of each of the stated objects, as well as objects of natural extension thereof, embodiments of the inventor's present system provide improved systems and methods for use of such system which will afford its recipients with one or more of the following benefits: (1) a higher incidence and degree of success in alleviating spinal deformities (in more dimensions of spinal column geometry than are presently addressed); (2) achievement of more nearly normal growth expectations; (3) the avoidance of some of multiple surgical procedures, associated discomfort and risks otherwise required in association with presently available spinal rod systems; (4) the elimination of a substantial degree of risk of spinal rod system component dislodgement; and (5) the maintenance of mobility at adulthood to a degree otherwise be lost though otherwise required fusions.

The spinal rod systems and the methods for use described herein, which are intended primarily to treat cases of juvenile scoliosis involving curvatures of greater than 45°, includes, in summary, adjustable length spinal rod, specifically an extendable telescopic spinal rod with means to slide or pass one end within another longitudinally, an anchor, for example a bone screw, more particularly a pedicle screw having a segment to be engaged to a bone or vertebra and a head segment to interface with the spinal rod, and a securing means configured for securing a mechanical engagement between the pedicle screw, for example, and the spinal rod for slidably engaging spinal rod[s] in a manner for both allowing passive, lengthwise adjustment while restraining axial rotation.

With respect to the latter feature, one embodiment of the present invention involves spinal rods of non-circular cross section that are telescopically engaged with complimentarily configured collar or sleeve members. This configuration (shown elsewhere herein) accommodates longitudinal, patient growth-related extension of the overall implant, while maintaining corrective orientation in multiple dimensions. Because of the complimentary contours of the non-circular spinal rods and the extendable telescopic spinal rod, a “slide-only engagement” is achieved. That is to say: longitudinal movement of the vertebrae is allowed with growth modulation rod system engaged, while at the same time axial rotation and other undesirable movement of the instrumented vertebrae relative to the spinal rod is substantially, or nearly completely arrested. Therefore, once the spinal rod is itself contoured according to the desired spinal geometry, optimal scoliotic correction (in three dimensions) is achieved, not only at the time of initial implantation, but is perpetuated as the patient grows. Further or future spinal longitudinal growth is modulated by control in three dimensions. As used herein, reference to “extendable” is meant to be the common definition, for example lengthening, elongating, or stretching. Also, telescopic or telescoping is meant to be slide or pass one within another or lengthwise movement with one part entering another as the result of elongating or compressing or one part sliding alongside the other part, for example as a U-shaped rod resting within another U-shaped rod.

Another embodiment of the present invention involves conventional, circular-in-cross-section spinal rods (usually two) that engage with telescoping collar, sleeve or bridge members to, once again, accommodate longitudinal, patient growth-related extension of the overall implant, while maintaining corrective orientation in multiple dimensions. In this latter case, axial rotation of the spinal rods relative to a collar, sleeve or bridge member is arrested, not by complimentary cross sectional geometry, but by fixed projections of various geometries that extend from the spinal rods through, and engage in a keyed fashion, with an elongate opening in the collar sleeve or bridge member(s). This configuration is also described and depicted elsewhere herein.

Optimal methods for achieving the initial scoliotic correction in three dimensions, which the present invention will maintain for the growing (juvenile) patient are best illustrated through reference to U.S. Patent Application, Publication No. 20060195092, which Application (and resulting Patent, if any) is hereby incorporated by reference.

### BRIEF DESCRIPTION OF THE DRAWINGS

The present invention may be more easily understood with reference to figures, which are as follow:

FIG. 1 is a diagrammatic, dorsal view of a spinal column with a growing spinal rod system of the present invention attached to selected vertebrae thereof.

FIG. 2 is a perspective depiction of an example of a pedicle screw having the unique spinal rod engagement means of the present invention for preventing axial rotation of the pedicle screw (and associated vertebrae) relative to the spinal rod.

FIG. 3 is a side elevational view of a spinal column having the preferred three pedicle screw “clusters” situated for engaging a spinal rod for the method of the present invention.

FIG. 4a is a diagrammatic, perspective view of the extendable telescopic spinal rod of the present invention, shown engaged with pedicle screw anchors as a non-circular cross sectional spinal rod in the “slide-only engagement” as an unextended position, and FIG. 4b as an elongated position, that is achievable, and is an object of the present growing rod spinal deviation correction system.

FIGS. 5a and 5b are diagrammatic, perspective view of the extendable telescopic spinal rod of the present invention, shown in various geometries for axial plane control of the growing rod spinal deviation correction system.

FIG. 6 is a side-by-side, elevational view of two spinal rod systems of the present invention (the latter-described embodiment), the system shown on the left in a shortened, pre-growth configuration, and the one on the right being shown in an expanded, post-growth configuration.

FIGS. 7a and 7b an elevational side view of a second embodiment, spinal rod system of the present invention, respectively, before and after elongation.

### DETAILED DESCRIPTION OF THE PREFERRED EMBODIMENT

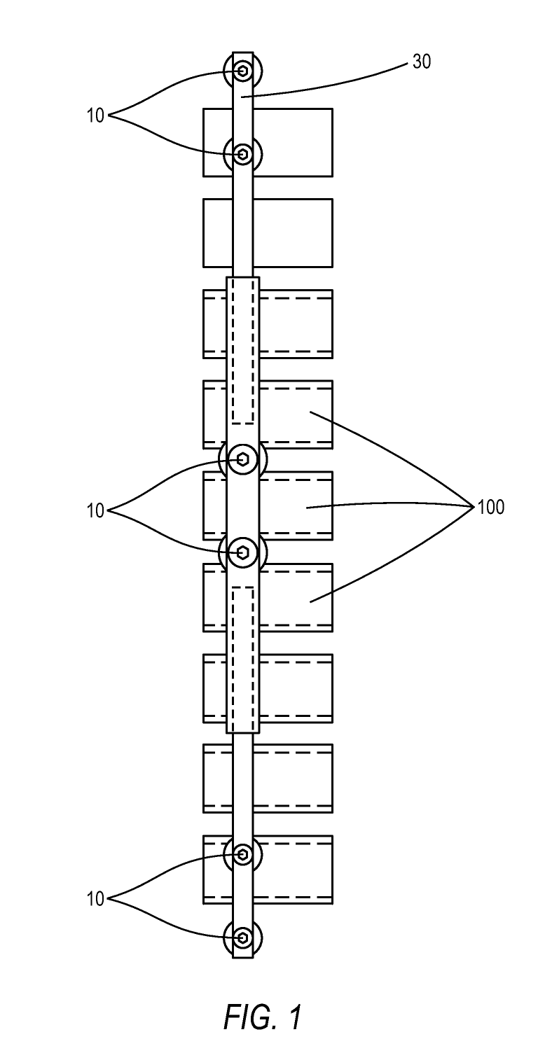

With reference to FIGS. 1-5b, the present growing rod spinal deviation correction system includes a number of pedicle screws 10, each implanted in respective vertebrae 100 to which forces will be applied by way of a properly contoured spinal rod 30, initially to achieve a scoliotic correction in an initial surgical intervention, and thereafter to maintain the desired correction, even as the patient grows.

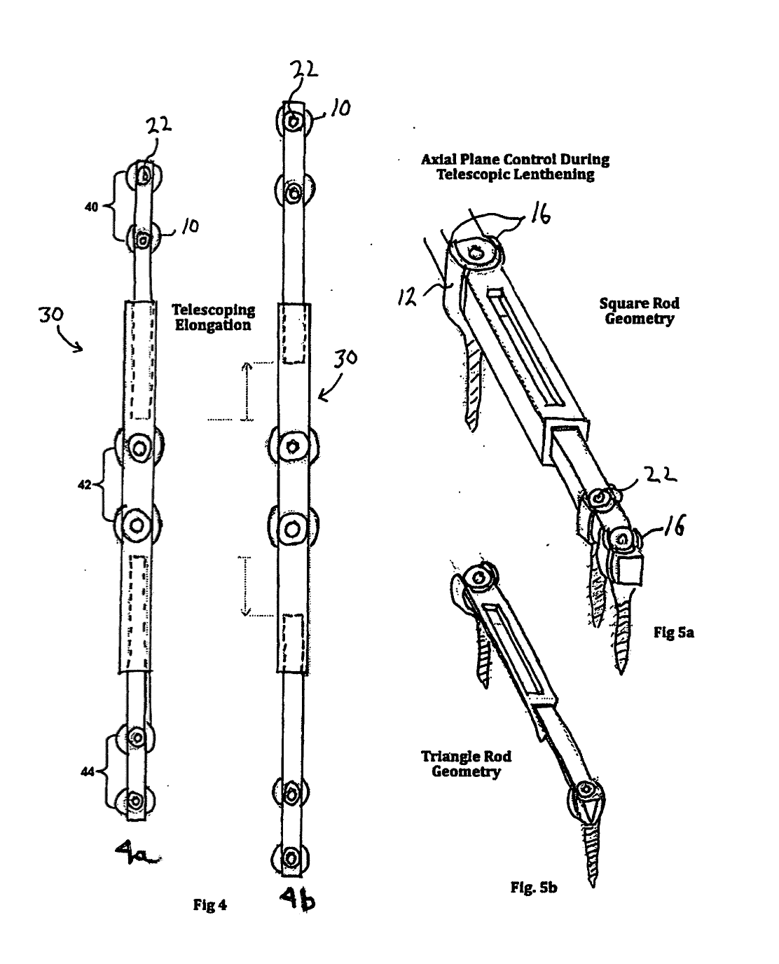

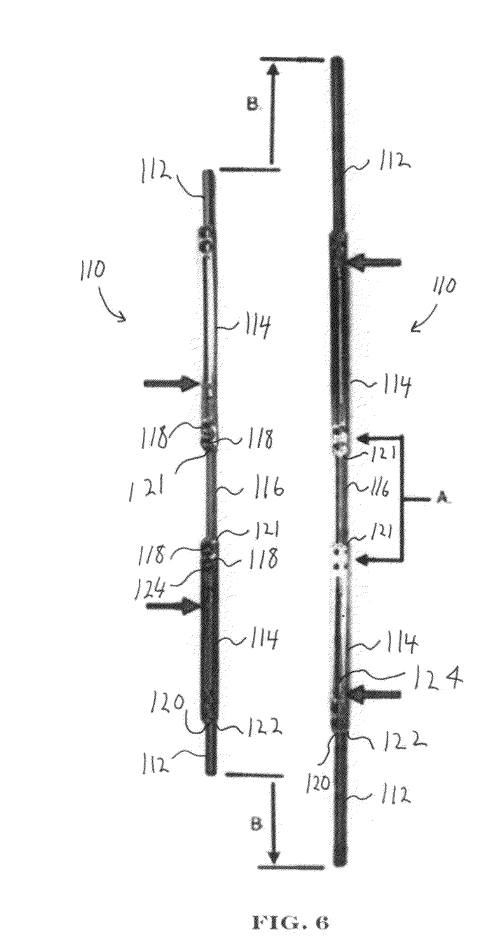

With particular reference to FIGS. 4a-4b and 5a-5b, pedicle screws 10 and spinal rod 30 are respectively configured such that spinal rod 30 is an adjustable length spinal rod, specifically an extendable telescopic spinal rod with a means to slide or pass one end within another may, in a “slide-only engagement,” slide longitudinally with movement (longitudinal growth) of the vertebrae 100 (and associated pedicle screw 10), but the same are constrained from any axial rotation and other undesirable movement because of the respective geometry of the spinal rod 30. and the portion of pedicle screws 10 with which the spinal rod 30 is mechanically linked (the “spinal rod engagement means”).

The depicted embodiment of spinal rod 30 shown in FIGS. 1, 3, 4a, 4b and 5a is of a substantially square cross sectional geometry and in FIG. 5b of a triangular cross sectional geometry, and the associated spinal rod engagement means is configured in a complimentary fashion for both: (1) allowing longitudinal movement of the spinal rod 30 relative to pedicle screws 10 and (2) preventing axial rotation and other undesirable movement of the pedicle screw 10 relative to spinal rod 30. However, it must be understood that other “non-circular” geometries for spinal rod 30 and the rod engagement means of pedicle screws 10 may be substituted for that shown herein as a preferred embodiment. For example cross sectional geometries (“non-circular geometries”) for spinal rods 30 may include (among others not listed) those which are triangular, hexagonal, rectangular, gear-toothed, cross-shaped, or ovoid, with the spinal rod engagement means portion of pedicle screws 10 being of a complimentary geometry. In each such case, by virtue of the relatively tight, nested engagement between a spinal rod 30 of non-circular cross sectional geometry with a spinal rod engagement means portion of pedicle screw(s) 10 of a complimentary geometry, substantially no axial rotation of pedicle screw 10 relative to spinal rod 30 is possible.

The extendable telescopic spinal rod 30 and pedicle screw 10 of the growing rod spinal deviation correction system may be made from any strong material such as carbon fiber or metal for long term sustainability. Preferred materials for spinal rod 30 may be, for example, stainless steel, or titanium, or chromium or alloy thereof, more particularly cobalt chromium or cobalt chromium molybdenum or alloy thereof, or other material known to one of skill in the art.

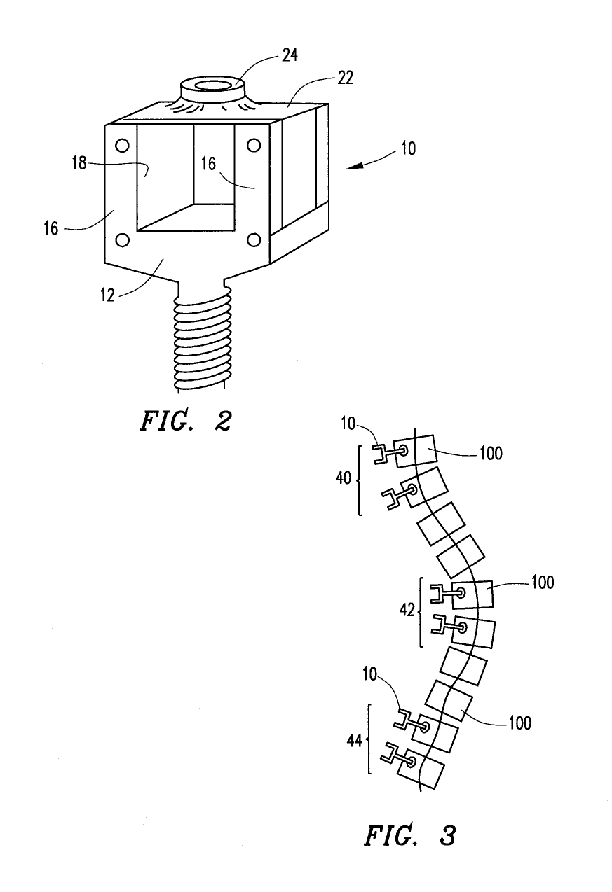

In the preferred embodiment of the pedicle screws 10 of the present invention, the head portion 12 of pedicle screws 10 is configured as a yoke-like structure for achieving a spinal rod engagement means, as depicted in FIG. 2. Two, upwardly projecting arms 16 cooperatively form this structure, defining a rod enclosure space 18, itself having a lateral opening 20 through which a segment of spinal rod 30 may be laterally introduced into the rod enclosure space 18.

A screw-in plug, or “set screw” 22 serves to occlude opening 20 and thereby constrain the associated length of spinal rod 30 within space 18. Set screw 22 is engaged in such a manner that it engages the adjacent surface of spinal rod 30 whereby substantially all relative movement between spinal rod 30 and pedicle screw 10 is arrested.

Referring particularly to FIGS. 1 and 3, the preferred method for use of the present growing rod system involves, by way of an example involving a right thoracic curve, placing pedicle screws 10 in three clusters. An upper cluster 40 involves two pedicle screws 10 placed in vertebrae 100 above the upper end vertebrae (“UEV” in FIG. 3) of the scoliotic curve; a middle cluster 42 placed in vertebrae 100 substantially at the apex of the scoliotic curve; and a lower cluster 44 placed in vertebrae 100 below the lower end vertebrae (“LEV” in FIG. 3) of the scoliotic curve. Generally a second growth modulation system of identical construction is placed on the opposite side of the midline to add strength to augment correction and prevent implant dislodgement. In certain embodiments, the upper cluster 40 and lower cluster 44 may serve as counter-rotation anchor points when the middle cluster 42 anchors the principal curve straightening and vertebral derotation correction.

Once spinal rod 30 is engaged with pedicle screws 10, and the initial three-dimensional scoliotic correction is achieved, plugs or set screws are engaged with each of the pedicle screws 10, and are tightened to “anchor” spinal rod 30, while the extendable telescopic spinal rod allows the earlier-described longitudinal movement of the spinal rod with the vertebrae and associated pedicle screws 10. Accordingly, as the spinal column grows or the distance in spinal segments increases, the extendable telescopic spinal rod elongates in the same plane relative to the longitudinal growth of the vertebrae and associated pedicle screws, providing for relatively uninhibited growth of the spinal segments.

The extendable telescopic spinal rod 30 of the growth modulation rod spinal deviation correction system provides a large piston-cylinder sliding (extendable telescoping rod) smooth surface area interface, greatly improving the operability of the adjustable rod. The larger surface area interface for the extendable telescopic spinal rod 30 also reduces the chance for wear of the system parts, particularly metal wear and scoring that could lead to binding and possible metal debris and ion release. Metal wear and binding may occur in particular in other systems in which pedicle screws are engaged to slide longitudinally along a spinal rod.

Once the present spinal rod system is implanted, as described, a juvenile patient's subsequent growth is unhindered by the system, while correction of the scoliotic curve is maintained to maturity and thereafter. Proper relative alignment of the vertebrae is maintained, as is the individual orientation of affected vertebrae, thereby achieving and maintaining a true three-dimensional scoliotic correction. Further or future spinal longitudinal growth is modulated by control in three dimensions.

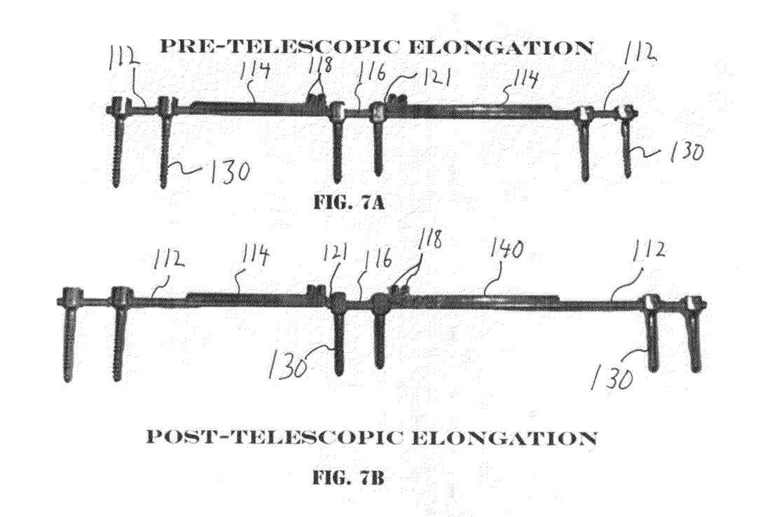

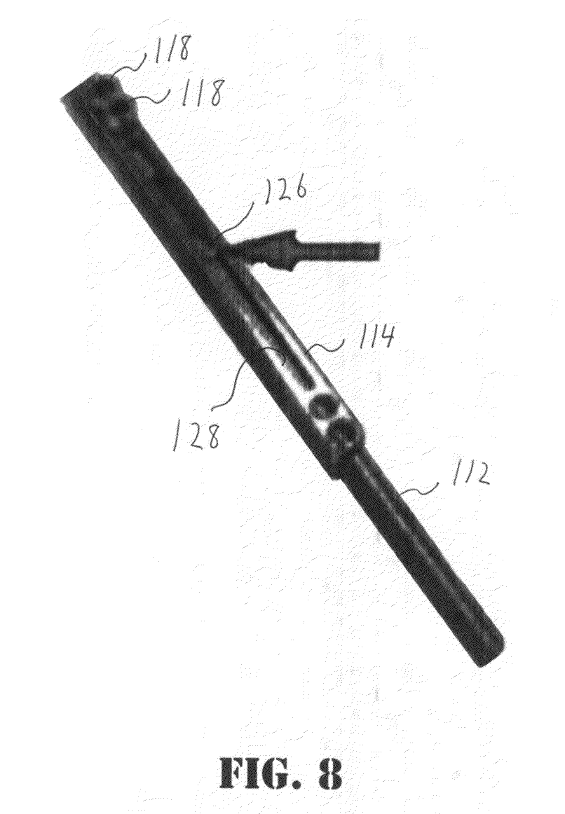

A second embodiment of the present invention of a growth-accommodating, three-dimensional correction spinal rod system is depicted in FIGS. 6, 7a and 7b. The spinal rod systems of the second embodiment are identified generally by the reference number 110.

Each system 110 includes at least two spinal rods 112, one or more sleeve, collar or bridge members (hereafter “collar member”) 114, and a central rod member 116. The anticipated, optimal structure involves two collar members 114 that are rigidly attached respectively to each end of the central rod member 116—held in-place by setscrews 118.

Each collar member 114 is attached at its medial end 121 to the central rod member 116, and defines a channel 120 into which, on a lateral end 122 of collar 114, a medial end 124 of a spinal rod 112 is telescopically received.

Referring to FIG. 8. a projection 126 extends from the surface of each spinal rod 112, extending through and slidably engaging a longitudinal slot 128 formed in each collar member 114. This arrangement permits longitudinal movement of each spinal rod 112 relative to each collar member 114, while resisting axial rotation of the spinal rod 112 relative to the collar 114. This, in turn, has the effect if imparting corrective and axial rotational constraining forces on the subject spinal column to which system 110 is attached (by way of pedicle screws 130), as the rod, with respect to three dimensions as discussed above, will have been contoured to effect the desired spinal correction, leaving only elongation of the system 110 for accommodating growth as nearly the sole remaining free motion of relative system components.

Regardless of the embodiment of the present invention that is chosen, constraint of relative movement of spinal rod systems of the present invention, excepting only longitudinal, over-all system length, achieves each of the objects stated above. Users can expect: (1) a higher incidence and degree of success in alleviating spinal deformities (in more dimensions of spinal column geometry than are presently addressed); (2) achievement of more nearly normal growth expectations; (3) the avoidance of some of multiple surgical procedures, associated discomfort and risks otherwise required in association with presently available spinal rod systems; (4) the elimination of a substantial degree of risk of spinal rod system component dislodgement; and (5) the maintenance of mobility at adulthood to a degree that would otherwise be lost through otherwise required fusions.

Although the invention has been described with reference to specific embodiments, this description is not meant to be construed in a limited sense. Various modifications of the disclosed embodiments, as well as alternative embodiments of the inventions will become apparent to persons skilled in the art upon reference to the description of the invention. It is, therefore, contemplated that the appended claims will cover such modifications that fall within the scope of the invention.

1. An improved spinal rod system comprising:
an extendable telescopic spinal rod having first and second telescopically engaged rod component means, said first rod component means being telescopically receiving within an end of said second rod component means, said first rod component means having first engagement means and said second component means having second engagement means, said first and said second engagement means being respectively configured to engage with each other for permitting, while engaged, said first and said second rod component means to move relative to each other substantially along their respective longitudinal axes, and for substantially resisting axial rotation of both said first and said second rod component means; and
a plurality of pedicle screws, each said pedicle screw having spinal rod engagement means for secure engagement with said extendable telescopic spinal rod.

2. The system of claim 1 wherein said spinal rod engagement means comprises constituents of a head portion of said pedicle screw which define a spinal rod enclosure space which is configured in such a manner as to engage positively a segment of said spinal rod in a manner for preventing relative movement of said pedicle screw and said spinal rod in all directions.

3. An improved spinal rod system comprising:
an extendable telescopic spinal rod having a non-circular cross sectional geometry;
a plurality of pedicle screws, each said pedicle screw having spinal rod engagement means, and
a telescopic spinal rod allowing for longitudinal movement, while resisting axial vertebral rotation with a securing means configured for interfacing with said pedicle screw and thereafter for securing a mechanical engagement between a said segment of said telescopic spinal rod and said spinal rod engagement means.

4. The system of claim 3 wherein said spinal rod engagement means comprises constituents of a head portion of said pedicle screw which define a spinal rod enclosure space which is contoured in such a manner as to engage positively a segment of said spinal rod in a manner for preventing relative movement of said pedicle screw and said spinal rod in all directions.

5. The system of claim 4 wherein said spinal rod exhibits a non-circular cross sectional geometry, or a rigid keyed geometry between telescopic spinal rod system piston and cylinder components which resists axial rotational deviation while modulating longitudinal spinal column growth.
